(19) United States
(12) Patent Application Publication
(10) Pub. No.: US 2025/0082405 A1
(43) Pub. Date: Mar. 13, 2025

(54) SINGLE FIBER, EXCIMER LASER, LESION CROSSING SYSTEM

(71) Applicant: KONINKLIJKE PHILIPS N.V., EINDHOVEN (NL)

(72) Inventor: Ryan Michael SOTAK, COLORADO SPRINGS, CO (US)

(21) Appl. No.: 18/580,645

(22) PCT Filed: Aug. 16, 2022
(86) PCT No.: PCT/IB2022/000505

§ 371 Date: Jan. 19, 2024

Related U.S. Application Data
(60) Provisional application No. 63/228,669, filed on Aug. 3, 2021.

(51) Int. Cl. A61B 18/24; A61B 17/00; A61B 17/3207; A61B 18/00; A61B 18/22

(52) U.S. Cl. CPC A61B 18/245; A61B 17/320758; A61B 2017/00778; A61B 2018/00107; A61B 2018/00404; A61B 2018/00577; A61B 2018/2205

(57) ABSTRACT

A vascular lesion crossing device (10) includes a single optical fiber or optical fiber bundle (12) configured for insertion through a lesion in a blood vessel, the single optical fiber or optical fiber bundle being couplable to an associated laser generator (18). The single optical fiber or optical fiber bundle has a diameter of 1.5 mm or less.

### FIELD

The following relates generally to the catheter arts, catheter guidewire arts, vascular therapy, lesion treatment arts, and related arts.

### BACKGROUND

In catheter-based vascular therapy, a catheter bears one or more tools at its distal end, such as an angioplasty balloon, a laser aperture or cutting tool for thrombectomy or atherectomy, a stent and associated stent deployment hardware, and/or so forth. Initially, a guidewire is inserted into a blood vessel and is fed out until the guidewire crosses past a treatment area (for example, a clot, thrombus, aneurysm, or so forth). The catheter has a guidewire lumen and is inserted along the guidewire into the blood vessel to move the catheter tip to the treatment area. However, total (or near total) occlusions within vasculature (e.g., arteries or veins) are very difficult to cross. This may cause the operator (e.g., physician or surgeon) to poke outside of a main lumen of the vessel to get around the occlusion. If the occlusion is able to be crossed, it generally takes an extremely long time in order for a guidewire to slowly push through the blockage. Additionally, the occlusions comprise a stronger material than the vessel wall, meaning that it is easy to cause a rupture in the vessel while attempting to cross the occlusion, which would require additional intervention to fix. Moreover, in some cases, a physician is completely unable to cross the occlusion, which and catheter-based procedure is then aborted.

Crossing of intravascular lesions is often the most time-consuming portion of an intravascular procedure. This can typically take 30-90 minutes to execute, even in experienced hands. In inexperienced hands, it often leads to procedure failure.

The following discloses certain improvements to overcome these problems and others.

### SUMMARY

In some embodiments disclosed herein, a vascular lesion crossing device includes a single optical fiber or optical fiber bundle configured for insertion through a lesion in a blood vessel, the single optical fiber or optical fiber bundle being couplable to an associated laser generator. The single optical fiber or optical fiber bundle has a diameter of 1.5 mm or less.

In some embodiments disclosed herein, a vascular therapy method includes: inserting an optical fiber or fiber bundle through a blood vessel to position an end of the optical fiber or fiber bundle at a lesion in the blood vessel; energizing, with a laser generator, the optical fiber or fiber bundle to form an opening in the lesion at least in part by laser ablation using light from the laser generator that passes through the optical fiber or fiber bundle; inserting a vascular therapy device which is different from the optical fiber or fiber bundle through the blood vessel to position the vascular therapy device at the lesion in the blood vessel; and performing a therapy on the lesion in the blood vessel using the vascular therapy device.

In some embodiments disclosed herein, a vascular lesion crossing device includes an optical fiber configured for insertion through a lesion in a blood vessel, the optical fiber being couplable to an associated laser generator; and a support catheter insertable into the blood vessel and having a central lumen sized to pass the optical fiber.

One advantage resides in providing a guidewire insertion device and corresponding guidewire insertion method providing efficient and safe guidewire crossing of a vascular obstruction or lesion.

Another advantage resides in providing such a guidewire insertion device with a support sheath to increase an ease of pushing of the insertion device through a lesion.

Another advantage resides in energizing a guidewire insertion device to allow the insertion device to cross a lesion.

Another advantage resides in reducing an amount of time needed for a guidewire insertion device to cross a lesion or obstruction.

A given embodiment may provide none, one, two, more, or all of the foregoing advantages, and/or may provide other advantages as will become apparent to one of ordinary skill in the art upon reading and understanding the present disclosure.

### BRIEF DESCRIPTION OF THE DRAWINGS

The disclosure may take form in various components and arrangements of components, and in various steps and arrangements of steps. The drawings are only for purposes of illustrating the preferred embodiments and are not to be construed as limiting the disclosure.

FIG. 1 diagrammatically illustrates a crossing device in accordance with the present disclosure.

FIGS. 2A-2C diagrammatically illustrate other embodiments of the single optical fiber or fiber bundle of the crossing device of FIG. 1.

FIG. 3 diagrammatically illustrates the crossing device of FIG. 1 at the point in which the single optical fiber or fiber bundle has formed a crossing penetration through the lesion.

FIG. 4 diagrammatically illustrates a vascular therapy stage subsequent to the point in time of FIG. 3, in which the single optical fiber or fiber bundle has been withdrawn and a guidewire inserted through the crossing penetration through the lesion.

FIG. 5 diagrammatically illustrates a vascular therapy stage subsequent to the point in time of FIG. 4, in which a vascular therapy device has been inserted into the blood vessel along the guidewire and positioned to treat the lesion.

FIG. 6 diagrammatically illustrates a method of performing a vascular therapy method using the device of FIG. 1.

### DETAILED DESCRIPTION

In intravascular procedures to treat lesions in blood vessels, a thin guidewire is initially inserted and run up to and through the lesion. Thereafter, a therapeutic device is inserted up to the lesion, with the guidewire running through a central lumen of the therapeutic device. The therapeutic device is then operated to treat the lesion, for example by removal (cutting with a rotary cutter, ablating using an excimer laser, or so forth) or by remodeling using an angioplasty balloon or the like.

The passage of the guidewire through the intravascular lesion is referred to as crossing the lesion. This is typically done by mechanical force, performed by a skilled vascular surgeon; nonetheless, the crossing can take well over an hour, and in some instances cannot be achieved at all, leading to aborting of the intravascular procedure. Crossing the lesion using the guidewire typically relies on a piercing/sheering force applied by the tip of the guidewire to mechanically separate the lesion and allow passage. When the lesion surface is not flat or perpendicular to the trajectory of the guidewire or other crossing tool, it can deflect and lead to vessel perforation, or entering the sub-intimal plane.

The following discloses an approach in which the lesion crossing is done using an optical fiber of diameter comparable with or slightly larger than the guidewire, e.g., a diameter of 0.05-inches or less in some contemplated embodiments. The optical fiber is inserted to position an end of the single optical fiber or fiber bundle (specifically, the distal end of the fiber or fiber bundle which serves as a light output end) at a lesion in the blood vessel then energized using an excimer laser operating at 308 nm, an ultraviolet (UV) laser operating at 355 nm, or another type of ablative laser. Advantageously, the laser can be identical with the laser generator used for driving a therapeutic device that employs laser ablation.

As the optical fiber is not required to have significant mechanical strength (as it is not used to physically punch through the lesion), it may be fed in through a support catheter. In this approach, the support catheter is inserted until it reaches the lesion, then the optical fiber is inserted through the support catheter to extend a few millimeters out of the support catheter to optically engage the lesion to perform the crossing by laser ablation. After the optical fiber and support catheter cross the lesion by way of laser ablation (optionally in conjunction with auxiliary mechanical pressure that can be provided using the support catheter), the optical fiber is withdrawn from the support catheter and the usual guidewire is inserted through the support catheter and through the lesion via the laser-ablated opening produced by the optical fiber. The support catheter is then withdrawn, and the therapeutic device inserted with the guidewire passing through its central lumen to reach the lesion, and the therapy is performed as usual.

In some embodiments disclosed herein, a single fiber is disclosed, which has advantages including providing a more concentrated and uniform laser beam for ablation to cross the lesion and optionally being couplable to the laser generator using a quick connect/disconnect iris coupling. However, in other embodiments, the optical fiber could be permanently mechanically fixed to a coupling device. The optical fiber could also be a fiber bundle without a central lumen.

While primarily described herein for the crossing of intravascular lesions in peripheral arteries and veins, the disclosed systems and methods could also be applied for crossing lesions in other types of vasculatures, such as cardiac and neurovascular vessels.

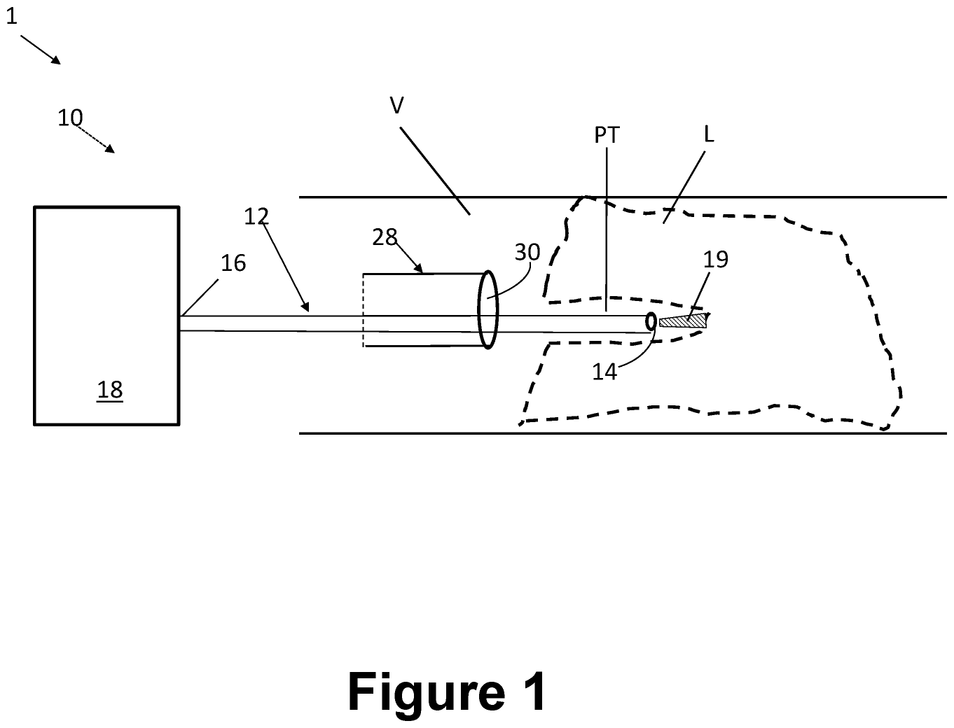

With reference to FIG. 1, an illustrative lesion crossing apparatus 1 is diagrammatically shown. As shown in FIG. 1, the apparatus 1 includes a single optical fiber or fiber bundle 12 that is insertable into a vascular therapy device 10 insertable into a blood vessel V for treating a lesion L (or a clot, or an occlusion, and so forth) in the blood vessel V. The crossing device 10 can comprise, for example, a single optical fiber or fiber bundle 12 configured for insertion through a lesion in a blood vessel. The optical fiber 12 has a diameter of 1.5 mm or less (e.g., ranging from 0.356 mm-0.889 mm in some embodiments). The optical fiber 12 includes a distal end 14 serving as a light output aperture 14, and a proximal end 16 operatively coupled to a laser generator 18. The laser generator 18 is configured to energize the optical fiber 12 with a laser beam that passes through the single optical fiber or fiber bundle 12 and exits the light output end 14 of the fiber 12 to treat the lesion L.

Notably, the single optical fiber or fiber bundle 12 is a lesion crossing device. It is not a vascular therapy device such as a laser ablation therapy device or a lesion cutting device of the type used in a thrombectomy or atherectomy. The single optical fiber or fiber bundle 12 has a small diameter of 1.5 mm or less, and cannot feasibly be used to perform a thrombectomy or atherectomy because a laser light 19 output from the distal end 14 of the single optical fiber or fiber bundle 12 has a small diameter comparable with the vessel diameter, and can only form penetration PT into the lesion L of a comparably small-diameter. (FIG. 1 is diagrammatic and is not drawn to scale; the penetration PT is typically on the order 1-2 mm or less in diameter). By contrast, a laser ablation catheter of a type used in a vascular therapy such as thrombectomy or atherectomy typically has a fiber bundle of a larger diameter to carry high power delivered over a large cross-sectional area. The fiber bundle of a laser ablation catheter also typically has an inner lumen for receiving a guidewire. By contrast, the single optical fiber or fiber bundle 12 is a single optical fiber, or at most a fiber bundler with a narrow diameter of 1.5 mm or less.

On the other hand, the large diameter of the optical fiber bundle of a laser ablation catheter, along with the typical requirement to be fed into the blood vessel along a guidewire, means that a laser ablation catheter typically cannot be used to perform the initial crossing of the lesion in order to feed the guidewire through the lesion. By contrast, the single optical fiber or fiber bundle 12 with its small diameter comparable to or slightly larger than the diameter of a typical guidewire (e.g., ranging from 0.356 mm-0.889 mm for some standard guidewire diameters) is effective for crossing the lesion L, that is, for creating the narrow-diameter penetration PT that is insufficient to constitute clinical treatment of the lesion L (as the narrow-diameter penetration PT is insufficient to carry substantial blood flow) but is suitable for subsequently passing a guidewire through the lesion L.

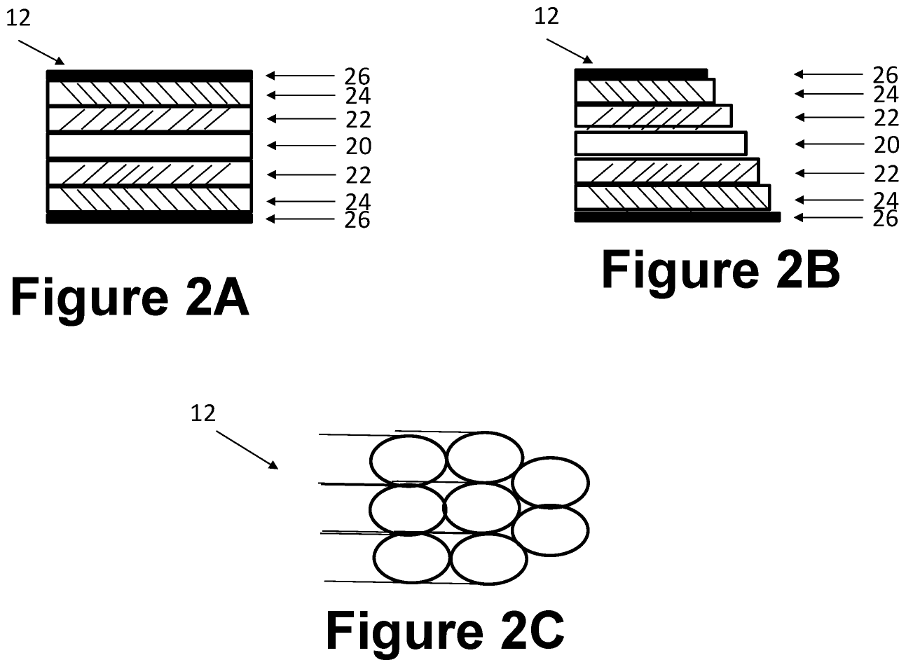

Referring now to FIGS. 2A-2C, and with continuing reference to FIG. 1, different embodiments of the single optical fiber or fiber bundle 12. FIG. 2A shows a side view of the optical fiber 12 (in this embodiment constituting a single optical fiber) with the optical output end 14 comprising a square tip, while FIG. 2B shows the optical fiber 12 (again as a single optical fiber) with the optical output end 14 comprising a beveled tip. FIGS. 2A and 2B shows layers comprising the optical fiber 12. A core of the optical fiber 12 comprises a glass core layer 20. A cladding layer 22 surrounds the glass core layer 20, and a polyimide buffer layer (or similar material) 24 surrounds the cladding layer 22. In some embodiments, a hydrophilic coating 26 can surround the polyimide buffer layer 24.

By contrast to the embodiments of FIGS. 2A-2B in which the single optical fiber or fiber bundle 12 comprises a single optical fiber, FIG. 2C shows an embodiment in which the single optical fiber or fiber bundle 12 comprises an optical fiber bundle that (unlike a typical laser ablation catheter) does not have an internal lumen. Each optical fiber 12 of the bundle can have any of the properties shown in FIGS. 2A-2B (e.g., a square tip, a beveled tip, the layers 20, 22, 24, 26 shown in FIG. 2B, and so forth).

Referring back to FIG. 1, the crossing device 1 further includes a support catheter 28 insertable into the blood vessel V and configured to deliver the single optical fiber or fiber bundle 12 to the lesion L. (Note, diagrammatic FIG. 1 illustrates only the distal end of the support catheter 28, but it is fed into a blood vessel penetration and through the blood vessel V to position its end at the lesion L as shown in FIG. 1). The support catheter 28 includes a central lumen 30 that is sized to pass the optical fiber 12 to the lesion L. A vascular therapy device 32 that is different from the optical fiber 12 (e.g., a cutter, an ablation laser, a remodeling device, and so forth) is also configured to be delivered to the lesion L insertable into the blood vessel via the support catheter 12 to treat the lesion L.

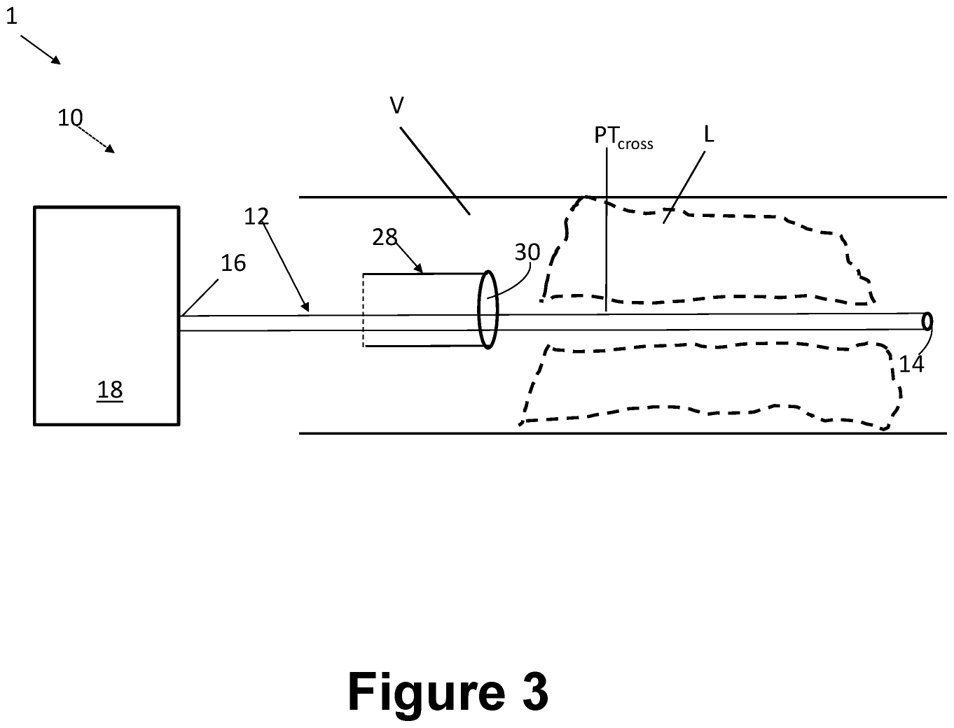

With reference to FIG. 3, the crossing device 10 of FIG. 1 is shown again, but now with the single optical fiber or fiber bundle 12 having laser-ablated the penetration PT of FIG. 1 to form an extended crossing penetration PT_{cross} that passes all the way through the lesion L. FIG. 3 thus shows the point at which the lesion L has been crossed. Notably, the lesion L has not yet been clinically treated, as the crossing penetration PT_{cross} is only around 1-2 mm in diameter and does not provide therapeutically acceptable blood flow.

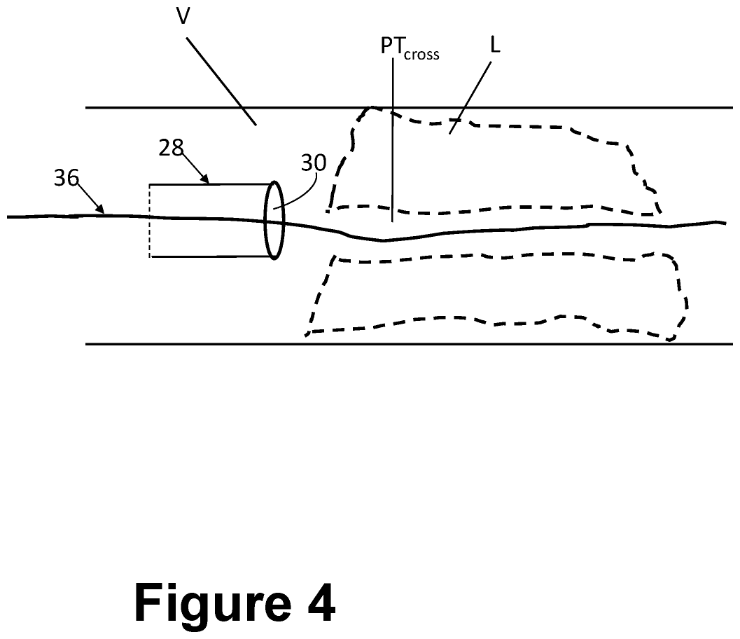

With reference to FIG. 4, after the lesion L has been crossed as shown in FIG. 3, the single optical fiber or fiber bundle 12 is withdrawn from the support catheter 28, and a guidewire 36 is inserted through the support catheter 28 and through the crossing penetration PT_{cross} so that the guide wire as shown in FIG. 4 is now crossing the lesion L. In this illustrative example, the same support catheter 28 is used for inserting the guidewire 36 as was used to insert the single optical fiber or fiber bundle 12; however, in an alternative embodiment the support catheter 28 may also be withdrawn prior to insertion of the guidewire 36, and in this alternative embodiment the guidewire 36 is inserted through the blood vessel L and through the crossing penetration PT_{cross} without using the support catheter 28.

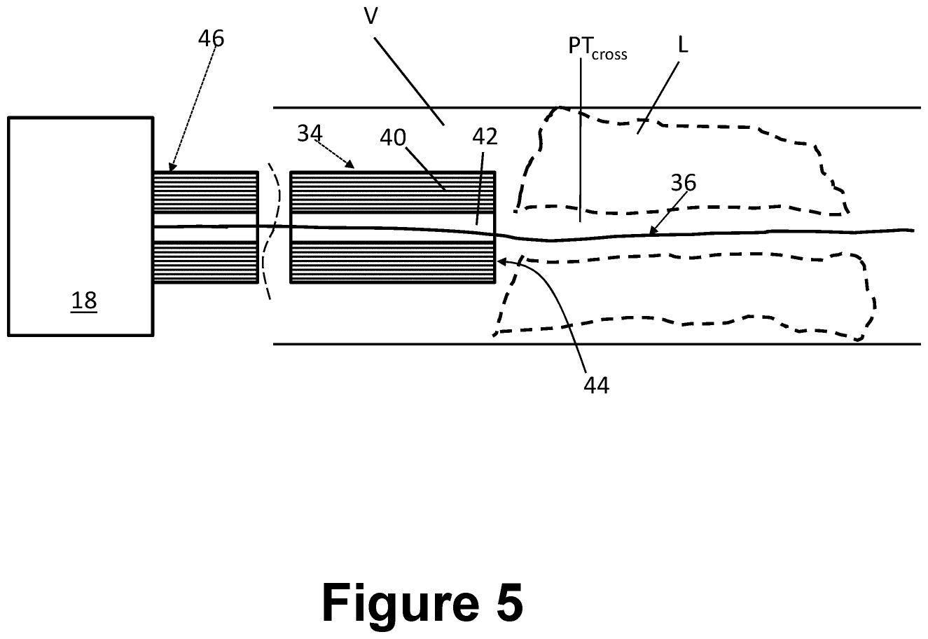

With reference to FIG. 5, a vascular therapy device 34, which is different from the single optical fiber or fiber bundle 12, is inserted through the blood vessel along the guidewire 36 to position the vascular therapy device 12 at the lesion L in the blood vessel V. The illustrative vascular therapy device 34 is a laser ablation catheter 34 having an annular optical fiber bundle 40 surrounding a central lumen 42 through which the guidewire 36 passes. The annular optical fiber bundle 40 has a distal end 44 proximate to the (now-crossed) lesion L for outputting laser light to ablate the lesion L to perform a thrombectomy, atherectomy, or other vascular therapy procedure. A proximal end 46 of the laser ablation catheter 34 suitably connects to the same laser generator 18 that was used to drive the single optical fiber or fiber bundle 12 to perform the crossing as previously described with reference to FIGS. 1 and 3. However, the larger diameter of the annular optical fiber bundle 40 compared with the smaller (e.g. 1.5 mm or smaller) diameter of the single optical fiber or fiber bundle 12 enables the laser ablation catheter 34 to deliver more optical power over a larger cross-sectional area in order to remove a therapeutically useful amount of the lesion L so as to substantially restore blood flow through the blood vessel V.

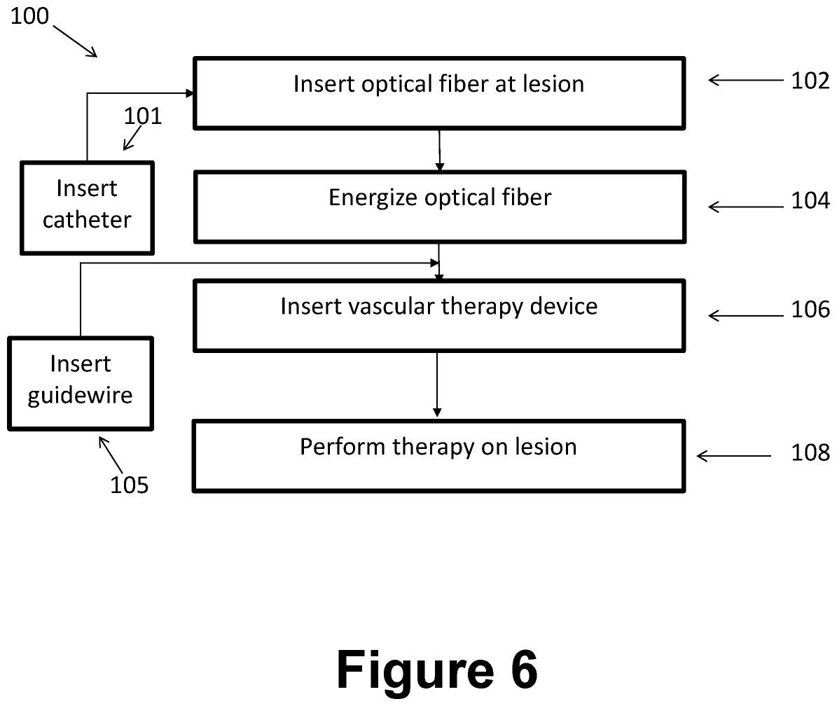

Referring to FIG. 6, an illustrative embodiment of a vascular therapy method 100 using the vascular therapy apparatus 1 is diagrammatically shown as a flowchart. At an optional embodiment 101, the support catheter 28 is inserted through the blood vessel V and positioning an opening where the central lumen 30 terminated at the lesion L. At an operation 102, the optical fiber 12 is inserted through the blood vessel V to position the light output end 14 of the optical fiber 12 at the lesion L in the blood vessel V. When the operation 101 is performed, the optical fiber 12 is inserted through the central lumen 30 of the support catheter 28 to arrive at the lesion L.

At an operation 104, the laser generator 18 is configured to energize the optical fiber 12 to form an opening (i.e., the penetration PT) in the lesion L with a laser beam. For example, the laser beam can have a wavelength ranging from 308 nm to 355 nm to energize the optical fiber 12.

At an optional operation 105, once the opening is formed in the lesion L, the optical fiber 12 and/or the support catheter 28 can be retracted from the blood vessel V, and a guidewire 36 can be inserted through the blood vessel V through the opening in the lesion L.

At an operation 106, the vascular therapy device 34 is inserted through the blood vessel V to position the vascular therapy device 34 at the lesion L. In some embodiments, when the operation 105 is performed, the vascular therapy device 34 is inserted through the blood vessel V along the guidewire 36.

At an operation 108, a therapy is performed on the lesion L with the vascular therapy device 34. The therapy can include, for example, one or more of a cutting operation, a laser ablating operation, or a remodeling operation.

The disclosure has been described with reference to the preferred embodiments. Modifications and alterations may occur to others upon reading and understanding the preceding detailed description. It is intended that the exemplary embodiment be construed as including all such modifications and alterations insofar as they come within the scope of the appended claims or the equivalents thereof.

1. A vascular lesion crossing device, comprising:
a single optical fiber or optical fiber bundle configured for insertion through a lesion in a blood vessel, the single optical fiber or optical fiber bundle being couplable to an associated laser generator;
wherein the single optical fiber or optical fiber bundle has a diameter of 1.5 mm or less.

2. The vascular lesion crossing device of claim 1, wherein the single optical fiber or optical fiber bundle is a single optical fiber.

3. The vascular lesion crossing device of claim 2, wherein the single optical fiber has an optical output end comprising a square tip or a beveled tip.

4. The vascular lesion crossing device of claim 2, wherein the single optical fiber comprises:
a glass core layer;
a cladding layer surrounding the glass core layer; and
a polyimide buffer layer surrounding the cladding layer.

5. The vascular lesion crossing device of claim 2, wherein the single optical fiber comprises a hydrophilic coating.

6. The vascular lesion crossing device of claim 1, wherein the single optical fiber or optical fiber bundle is an optical fiber bundle that does not have an internal lumen.

7. The vascular lesion crossing device of claim 1, further comprising:
a support catheter insertable into the blood vessel and having a central lumen sized to pass the single optical fiber or optical fiber bundle.

8. A vascular therapy device comprising:
a vascular lesion crossing device of claim 1; and
a vascular therapy device which is different from the single optical fiber or optical fiber bundle of the vascular lesion crossing device and is configured to treat the lesion in the blood vessel.

9. A vascular therapy method, comprising:
inserting an optical fiber or fiber bundle through a blood vessel to position an end of the optical fiber or fiber bundle at a lesion in the blood vessel;
energizing, with a laser generator, the optical fiber or fiber bundle to form an opening in the lesion at least in part by laser ablation using light from the laser generator that passes through the optical fiber or fiber bundle;
inserting a vascular therapy device which is different from the optical fiber or fiber bundle through the blood vessel to position the vascular therapy device at the lesion in the blood vessel; and
performing a therapy on the lesion in the blood vessel using the vascular therapy device.

10. The vascular therapy method of claim 9, further comprising:
after forming the opening in the lesion, retracting the optical fiber or fiber bundle from the blood vessel, and inserting a guidewire through the blood vessel and through the opening in the lesion;
wherein the vascular therapy device is inserted through the blood vessel along the guidewire.

11. The vascular therapy method of claim 10, wherein the inserting of the optical fiber or fiber bundle includes:
inserting a support catheter through the blood vessel and positioning an opening of the support catheter at the lesion; and
wherein the optical fiber or fiber bundle is inserted through a central lumen of the support catheter.

12. The vascular therapy method of claim 11, further including:
removing the support catheter from the blood vessel; and
inserting, with the guidewire, the vascular therapy device to perform the therapeutic therapy on the lesion.

13. The vascular therapy method of claim 9, wherein the therapy comprises one or more of a cutting operation, a laser ablating operation, or a remodeling operation.

14. The vascular therapy method of claim 9, wherein the energizing comprises:
energizing the at least one optical fiber with a laser having a wavelength ranging from 308 nm to 355 nm.

15. A vascular lesion crossing device, comprising:
an optical fiber configured for insertion through a lesion in a blood vessel, the optical fiber being couplable to an associated laser generator; and
a support catheter insertable into the blood vessel and having a central lumen sized to pass the optical fiber.

16. The vascular lesion crossing device of claim 15, wherein the optical fiber has a diameter of 1.5 mm or less.

17. The vascular lesion crossing device of claim 15, wherein the optical fiber has an optical output end comprising a square tip or a beveled tip.

18. The vascular lesion crossing device claim 15, wherein the optical fiber comprises:
a glass core layer;
a cladding layer surrounding the glass core layer; and
a polyimide buffer layer surrounding the cladding layer.

19. The vascular lesion crossing device of claim 15, wherein the optical fiber comprises a hydrophilic coating.

20. A vascular therapy device comprising:
a vascular lesion crossing device as set forth in claim 15; and
a vascular therapy device which is different from the single optical fiber or optical fiber bundle and is configured to treat the lesion in the blood vessel.
